(19) United States
(12) Patent Application Publication
(10) Pub. No.: US 2022/0249156 A1
(43) Pub. Date: Aug. 11, 2022

(54) METHODS AND SYSTEMS FOR HAIR TREATMENT

(71) Applicant: SCITON, INC., PALO ALTO, CA (US)

(72) Inventors: David Boinagrov, Mountain View, CA (US); Rick MENEFEE, Palo Alto, CA (US); Andrew G. CARRILLO, Palo Alto, CA (US); Daniel K. Negus, Palo Alto, CA (US); James L. Hobart, Palo Alto, CA (US)

(73) Assignee: SCITON, INC., PALO ALTO, CA (US)

(21) Appl. No.: 17/729,345

(22) Filed: Apr. 26, 2022

Related U.S. Application Data
(63) Continuation of application No. 16/005,001, filed on Jun. 11, 2018, now abandoned.
(60) Provisional application No. 62/520,018, filed on Jun. 15, 2017.

(51) Int. Cl. A61B 18/14; A61B 34/30; A61B 90/00

(52) U.S. Cl. CPC A61B 18/1477; A61B 2018/00476; A61B 90/361; A61B 34/30

(57) ABSTRACT

Methods of treating hair are described herein, including methods of treating a hair follicle, a hair component, and/or a sebaceous gland. The method comprises imaging a target using one or more imaging devices, positioning one or more needles in a treatment position with respect to the target, providing electrical, optical, or chemical treatment to the target using the one or more needles.

### CROSS REFERENCE TO RELATED APPLICATIONS

This application claims priority pursuant to 35 U.S.C. § 119(e) to U.S. Provisional Patent Application Ser. No. 62/520,018, filed on Jun. 15, 2017, which is hereby incorporated by reference in its entirety.

### FIELD

The invention is generally related to methods and systems for treating hair, and, more specifically, methods and systems for removing hair.

### BACKGROUND

People have unwanted hair on different parts of their bodies: their face, back, arms, legs, underarms and other areas. These unwanted hairs can represent a cosmetic problem for both women and men. There are numerous options for hair removal: shaving, plucking, depilatory creams, hot waxing, laser or Broad Band intense pulse Light (BBL) hair removal, electrolysis, and other various methods. Some methods (shaving, plucking, depilatory creams, hot waxing) only result in the temporary removal of hair, often lasting only a few days or weeks. Other longer term options suffer from various drawbacks. For example, although laser and BBL treatments can result in stable long term, or permanent hair reduction, they are unable to treat patients with light hair (hair lacking enough eumelanin for effective treatment, e.g. blonde, red, light or grey hair). These treatments can also cause side effects associated with the application of the laser or BBL to skin, such as burns or hyper- or hypopigmentation (especially for darker skin types). Hair removal with electrolysis (or electrical epilation or electroepilation) has been shown to be effective on all types of hair color. However, since electrolysis is a manual procedure, the overall treatment is often very slow and the final results highly dependent on the skill of the electrologist. Given how widespread the problem of unwanted hair, improved methods of permanent hair removal are needed.

### SUMMARY

This disclosure describes methods and systems for hair treatment, including hair follicle treatment, hair component treatment, and/or sebaceous gland treatment and provides one or more advantages over other methods and systems. For example, the methods and systems described herein disclose a computer vision system for determining the location and orientation of the hair. The methods and systems herein further disclose a robotic mechanism to control the positional location and energy delivery (or an alternate method for structural damage or destruction) of the systems described here to perform fast and efficient removal of hair of any color. In addition, the treatment of sebaceous glands with electrolysis needle is described, as well as hair follicle and sebaceous gland laser treatment with an optical fiber. Additionally, the treatment of sebaceous glands and hair follicles with a robotic cannula is described. The robotic cannula can have a fluid delivery mechanism for hair reduction/removal or sebaceous gland destruction.

In an aspect, a system for hair treatment comprises a robotic needle system. Further, methods and systems described herein, in some embodiments, provide for the treatment of hair with an automated vision system (for hair location and orientation as well as a robotic electrolysis needle for placement of the needle, and for the application of energy to damage the hair structures for hair reduction and/or removal). A method or system described herein can also provide for the treatment of the sebaceous gland, including sebaceous gland destruction. Methods and systems described herein, in some instances, also include the use of robotic optical fibers and laser light, and/or a robotic cannula with a fluid delivery mechanism for hair reduction/removal or sebaceous gland destruction.

In one aspect, a method of treating hair comprises positioning one or more needles in a treatment position with respect to a target, and providing electrical, optical, or chemical treatment to the target using the one or more needles. In some cases, a target includes a hair follicle, hair component or a skin component, such as a sebaceous gland or a region of the target hair follicle adjacent to the sebaceous gland. In other cases, a target hair component includes a hair bulb or a hair bulge.

In another embodiment, a method further comprises removing a hair associated with the target hair follicle, the target hair component, and/or the skin component. For example, a target can first be treated by electrical, optical, or chemical treatment followed by removal of the hair associate with the target. In some cases, the hair is removed using robotic tweezers.

In another embodiment, a method further comprises imaging a target using one or more imaging devices. For example, a method comprises imaging a hair follicle, a hair component, and/or a skin component of a patient. The one or more imaging devices can comprise one or more cameras or systems of cameras. In other embodiments, the one or more imaging devices comprise one or more sensors, such as pressure sensors, light sensors, or other similar imaging device sensors.

In some embodiments, a method further comprises processing data received from the one or more imaging devices using a controller. In some cases, the controller comprises hardware and/or software capable of directing one or more actuators to position the one or more needles in the treatment position. The one or more actuators, in some embodiments, comprise one or more mechanical actuators. In other embodiments, the one or more actuators comprise or define a 6-axis robot.

In some embodiments, the one or more needles comprise one or more electrolysis needles. An electrical current is applied to the target using the one or more electrolysis needles.

In other embodiments, the one or more needles comprise one or more optical fibers. A laser light or Broad Band intense pulse Light (BBL) is applied to the target using the one or more optical fibers.

In still other embodiments, the one or more needles comprise one or more cannulas. A chemical treatment species is applied to the using the one or more cannulas.

In some cases, a method comprises attaching an applicator to the skin prior to providing electrical, optical, or chemical treatment. The applicator, in some instances, is attached to the skin with an adhesive, whereas in other instances, the applicator is attached to the skin with a vacuum or other suction mechanism.

In another aspect, a system comprises one or more needles for providing electrical, optical, or chemical treatment to a target hair follicle, hair component, and/or to skin component. A system, in other embodiments, comprises one or more imaging devices for imaging the target hair follicle, hair component, and/or skin component. In still other embodiments, a system comprises one or more actuators for positioning the one or more needles in a treatment position with respect to the target hair follicle, the target hair component, and/or to the skin component. The target skin component, in some instances, includes a sebaceous gland. The target hair component, in some instances, is a hair bulb or a hair bulge.

In some embodiments, the one or more actuators comprise one or more mechanical actuators. In other embodiments, the one or more actuators comprise or define a 6-axis robot.

In some cases, a system further comprises one or more controllers. A controller, for example, in some embodiments, comprises hardware and/or software for processing data received from the one or more imaging devices. In other embodiments, the controller comprises hardware and/or software for directing the one or more actuators to position the one or more needles in the treatment position.

In some embodiments, the one or more imaging devices comprise one or more cameras or systems of cameras. In other embodiments, the one or more imaging devices comprise one or more pressure sensors.

In some embodiments, the one or more needles of the system comprise one or more electrolysis needles. In these embodiments, the system further comprises an electric current source coupled to the one or more electrolysis needles. In some cases, part of the one or more electrolysis needles is coated with a non-conducting material and another part of the electrolysis needle is exposed.

In other embodiments, the one or more needles comprise one or more optical fibers. In these embodiments, the system further comprises a laser light or BBL source coupled to the one or more optical fibers.

In still further embodiments, the one or more needles comprise one or more cannulas. In these embodiments, the system further comprises a source of a chemical treatment species coupled to the one or more cannulas. The chemical treatment species, for example, can comprise a fluid containing caustic sodium hydroxide.

In other cases, a system further comprises an applicator configured to be attached to or disposed on the skin. An applicator, for example, can comprise a rectangular frame, wherein the rectangular frame is attached to the skin. In some instances, the rectangular frame is attached to the skin with an adhesive. In other instances, the rectangular frame is attached to the skin with vacuum or suction.

Still, in other cases, a system further comprises robotic tweezers for removing hair associated with the target hair follicle, the target hair component, and/or the additional target component.

These and other embodiments are described in more detail in the detailed description that follows.

### BRIEF DESCRIPTION OF THE FIGURES

The invention will now be described by way of example, with reference to the accompanying figures.

FIG. 1 illustrates a robotic electrolysis system and skin having hair.

FIG. 2 is a block diagram of a robotic electrolysis system.

FIG. 3A is an expanded view of a needle and a cross sectional view of a target.

FIG. 3B is an expanded view of a needle in a treatment position with respect to a target.

FIG. 3C is an expanded cross sectional view of the target and a needle after providing treatment to the target.

FIG. 4 is a side view of a 6-axis robotic system and skin having hair.

FIG. 5 is a side view of a robotic electrolysis system comprising an applicator attached to the skin.

FIG. 6A is an expanded cross sectional view of a target and a needle having robotic tweezers.

FIG. 6B is an expanded cross sectional view a robotic electrolysis system providing electrical treatment to a target.

FIG. 6C is an expanded cross sectional view of a target and a robotic electrolysis system with robotic tweezers ejecting from the skin.

FIG. 6D is an expanded cross sectional view of a robotic electrolysis system extending tweezers toward a target hair.

FIG. 6E is an expanded cross sectional view of tweezers of a robotic electrolysis system grasping a target hair by pinching.

FIG. 6F is an expanded cross sectional view of a robotic electrolysis system retracting pinched tweezers grasping a target hair.

FIG. 6G is an expanded cross sectional view of a robotic electrolysis system removing a target hair.

FIG. 7A is a side view of a needle having a fully exposed surface.

FIG. 7B is a side view of a needle having a partially exposed surface.

FIG. 7C is a side view of a needle having a partially exposed surface.

FIG. 8A is an expanded cross sectional view of a needle providing treatment to a sebaceous gland.

FIG. 8B is an expanded cross sectional view of a needle providing treatment to a hair bulge.

FIG. 8C is an expanded cross sectional view of a needle providing treatment to a hair bulb.

FIG. 9 is an expanded cross sectional view of a needle comprising an optical fiber.

FIG. 10 is a block diagram of a robotic optical fiber system.

FIG. 11 is an expanded cross sectional view of a needle comprising a cannula.

### DETAILED DESCRIPTION

Embodiments described herein can be understood more readily by reference to the following detailed description and examples. Methods, devices, and features described herein, however, are not limited to the specific embodiments presented in the detailed description and examples. It should be recognized that these embodiments are merely illustrative of the principles of this disclosure. Numerous modifications and adaptations will be readily apparent to those of skill in the art without departing from the spirit and scope of the disclosure.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “from 5 to 10” or “5-10” should generally be considered to include the end points 5 and 10.

### I. Methods of Treating Hair

In one aspect, methods of treating hair are described herein, including methods of treating hair on human subjects or patients. Methods of treating hair can further include treating hair on various locations of a patient's body. Additionally, methods of treating hair include methods of treating hair follicles, hair components, and/or sebaceous glands located on the skin of a patient. Thus, methods of treating hair, as described herein, can include methods of treating any one or more macrostructures on or within the dermal layer of skin wherein hair grows. For example, methods of treating hair includes methods of treating the hair itself, hair follicle, papilla, hair matrix, root sheath, hair bulb, hair bulge, infundibulum, arrector pili muscle, sebaceous gland, apocrine sweat gland, hair follicle receptor, skin surface, dermal layer of the skin, region of a hair follicle adjacent to a sebaceous gland, and/or other related macrostructures identifiable on or within the region of skin wherein hair resides. Furthermore, any one or more of these macrostructures can be a target of one or more of the methods described herein.

In some embodiments, a method comprises positioning one or more needles in a treatment position with respect to the target at step 1202 of FIG. 12. The target, in some embodiments, can be a hair follicle, a target hair component, and/or an additional target component. Furthermore, the target, in other embodiments, can have a specific shape and/or size, including specific characteristics relative to its position in the skin, including visible surfaces on the outside of skin surface and hidden surfaces rooted within the dermal layer or epidermal layer of the skin. For example, a target can have a specific width, length, diameter, lean, directional growth, and/or depth into the skin. A target has a specific location and a spatial orientation relative to the surface of the skin. Thus, in some embodiments, one or more needles are positioned relative to these characteristics related to the position, location, and/or orientation of the target at step 1202. It should be understood that positioning the one or more needles in a treatment position at step 1202 includes positioning the one or more needles in preparation for puncturing the skin and inserting the one or more needles into the skin according the position, location, and/or orientation of the target.

Positioning of the one or more needles in a treatment position at step 1202, in some embodiments, further comprises inserting the one or more needles into the skin. For example, the one or more needles can, in some cases, be partially or fully inserted into skin. The needle, for example, can be inserted into the skin to a depth of at least 10 μm, at least 50 μm, at least 100 μm, at least 150 μm, between 10 μm and 10 mm, between 100 μm and 5 mm, between 100 μm and 2 mm, or between 100 μm and 1 mm. In some embodiments, the one or more needles are positioned in contact with the target. The position of the one or more needles in contact with the target, in some embodiments, are proximate, adjacent, or near the target. It should be understood that positioning the one or more needles in a treatment position at step 1202 includes positioning the one or more needles in any position on or within the skin, such that the target, located on or within the skin, is positioned to receive treatment from the needle.

In another embodiment, a method comprises providing electrical, optical, or chemical treatment to the target, as in step 3 at step 1203 of FIG. 12. The electrical, optical, or chemical treatment is, in some instances, provided directly to the target, such as the hair follicle, the hair component, and/or to the additional target component of the skin, upon contacting the target with the one or more needles. In some embodiments, providing electrical, optical, or chemical treatment to the target at step 1203 comprises heating a target and/or galvanizing a target.

Providing electrical, optical, or chemical treatment at step 1203, in some cases, functionally reduces, damages, or destroys the target, either permanently or temporarily, such that growth and/or re-growth of the target is prevented and/or slowed. In other cases, providing electrical, optical, or chemical treatment at step 1203 structurally reduces, damages, and/or destroys the target to remove or eliminate the macrostructure. In some instances, an additional target component can include a sebaceous gland or a region of the target hair follicle adjacent to the sebaceous gland wherein skin conditions, such as acne, can be prominent. In other instances, the target hair component can include a hair bulb or a hair bulge.

The amount, intensity, and/or time frame of the electrical, optical, or chemical treatment, in some embodiments, can be calculated and/or regulated. For example, treatment of the target can be based on the positioning of the one or more needles and further based on the characteristics and/or features of the target. For example, in some cases, additional treatment, such as by amount, intensity, time, or treatment type, may be performed for a particularly large, bulky, or otherwise difficult to treat target. Insofar as additional treatment is needed, it should be understood by a skilled artisan that methods described herein can be modified accordingly to obtain the goals of the method.

Wherein a method provides an electrical treatment to the target, the one or more needles of the method can comprise one or more electrolysis needles having conductive properties. In these embodiments, an electrical current is applied to the target, such as a hair follicle, a target hair component, and/or an additional target component, using the one or more electrolysis needles.

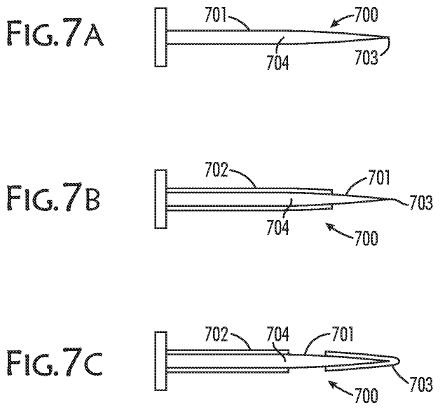

Furthermore, the electrolysis needle can have varying characteristics influencing the step of providing the treatment at step 1203. For example, an electrolysis needle 700 can have an exposed conductive surface 701 or a partially exposed and partially insulated or coated conductive surface 702. Electrolysis needles 700 having fully exposed surfaces 701, as shown in FIG. 7A, provide electrical treatment to all exposed surfaces 701 of the electrolysis needle 700, thereby providing energy to any target macrostructure in contact with or within reasonable distance of the exposed surface 701.

In other embodiments, an electrolysis needle 700 has a partially exposed conductive surface 701 and partially insulated or coated surface 702. For example, a partially exposed electrolysis needle 700, as shown in FIGS. 7B and 7C, can have one or more coatings of a non-conductive material, such as silicone or other suitable insulating polymer, providing insulating properties to the insulated or coated portions 702 of the electrolysis needle 700. Electrolysis needles 700 having one or more coatings still have a conductive core within the insulating coating. In some embodiments, the coatings partially cover the electrolysis needle 700, leaving a portion of the conductive core exposed 701. The exposed portion 701 of the electrolysis needle 700 can be positioned at any section of the electrolysis needle 700. In some embodiments, the tip 703 of the conductive electrolysis needle 700 is exposed and the rest of the electrolysis needle is coated, as shown in FIG. 7B. In other embodiments, a midsection 704 of the conductive electrolysis needle 700 is exposed and the rest of the electrolysis needle is coated, as shown in FIG. 7C. When a midsection 704 of an electrolysis needle 700 is exposed, the exposed surface 701 can include the entire circumferential surface area of a cross section of the electrolysis needle 700, or it can include only a fraction of the circumferential surface area of a cross section of the electrolysis needle 700. Electrolysis needles 700 having partially insulated surfaces 702 and partially exposed conductive surfaces 701 can provide energy to only the exposed surface 701 of the needle, rendering finer resolution of the electrical treatment. For example, methods providing electrical treatment wherein the electrical treatment is provided by one or more partially coated or partially insulated electrolysis needles 700, provide treatment through the exposed conductive surfaces 701 only, and only targets or macrostructures in contact with or within reasonable distance of the exposed conductive surfaces 701 are subjected to electrical treatment.

Wherein a method provides optical treatment to the target at step 1203, the one or more needles of the method can comprise one or more optical fibers 901. In these embodiments, a laser light or Broad Band intense pulse Light (BBL) is applied to the target, such as a hair follicle, a target hair component, and/or an additional target component using the one or more optical fibers 901.

Wherein a method provides chemical treatment to the target at step 1203, the one or more needles of the method can comprise one or more cannulas 1101. In these embodiments, a chemical treatment species is applied to the target, such as a hair follicle, a target hair component, and/or an additional target component using the one or more cannulas 1101. The cannula 1101, in some embodiments, is configured to deliver a liquid solution to the target. For example, in some instances, a liquid comprising sodium hydroxide, such as lye or caustic soda, is applied to the target.

Other needles can be used depending on the treatment type and the goals of the treatment. In some instances, more than one needle and/or needle type, can be used to provide electrical, optical, and/or chemical treatment to the target. For example, the one or more needles can be interchangeable. For example, a method described herein can position a first needle at step 1202 and provide a first treatment at step 1203, and subsequently position a second needle at step 1202, providing a second treatment at step 1203, wherein the first and second treatments are the same or different types of treatments. In some cases, an interchangeable needle having multiple needle types 102 is configured to provide one or more types of electrical, optical, and/or chemical treatment at step 1203. In other cases, the first needle type is disassembled from the electrolysis needle base prior to positioning the second needle type.

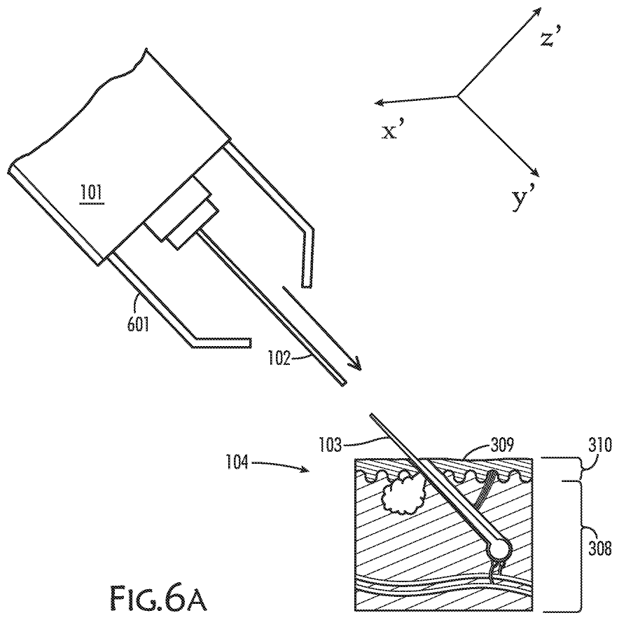

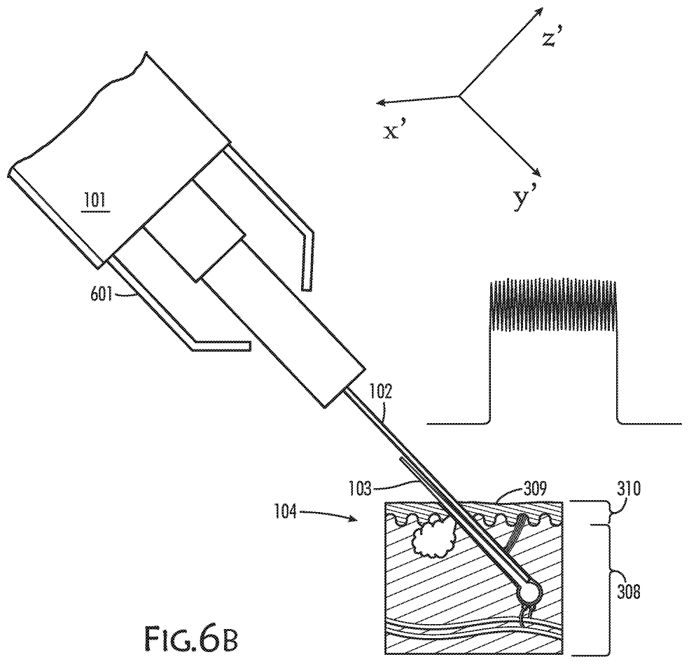

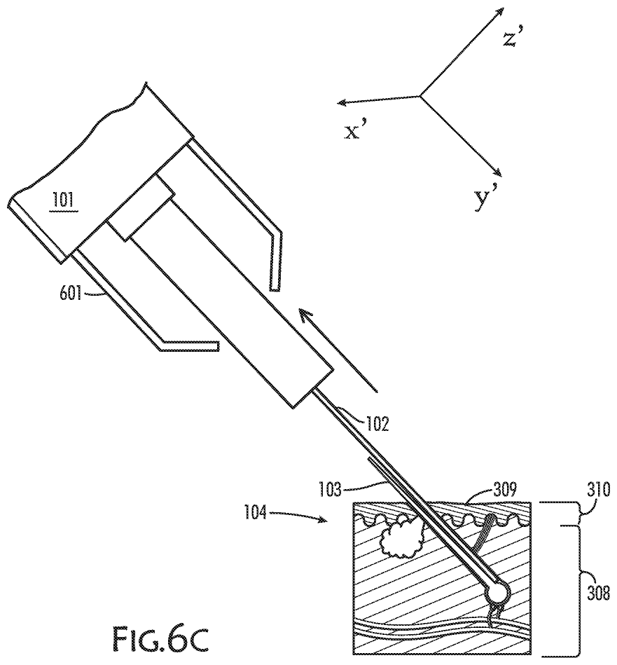

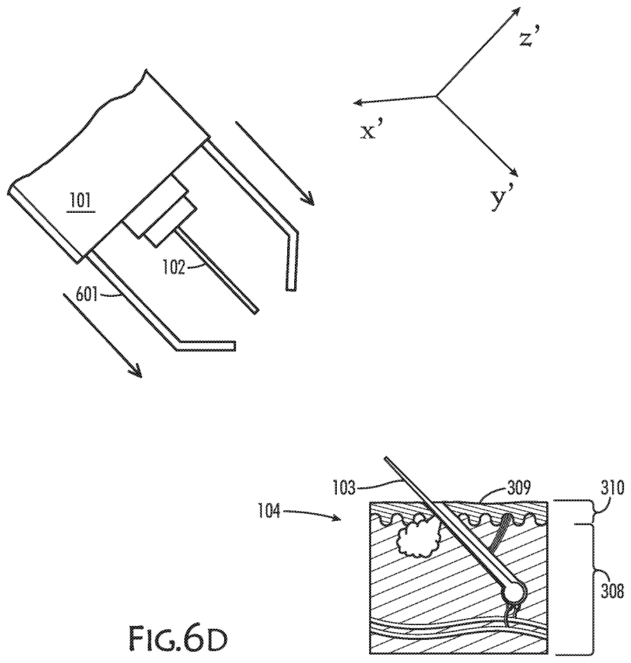

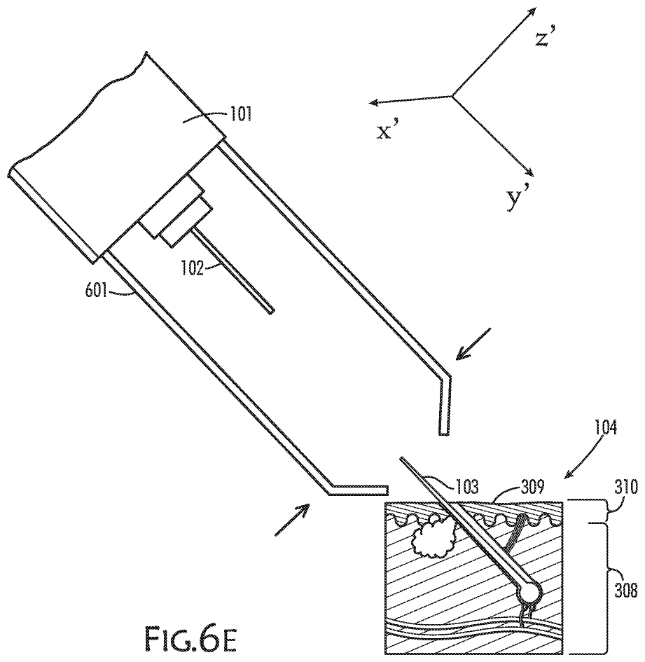

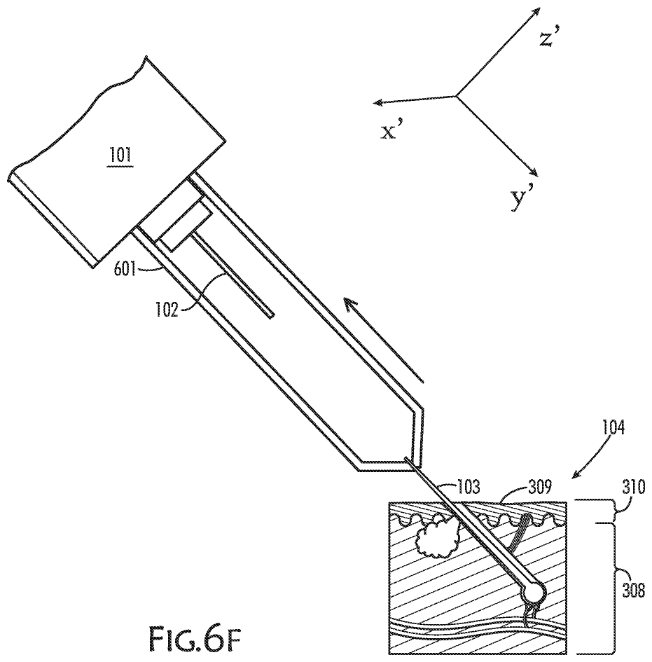

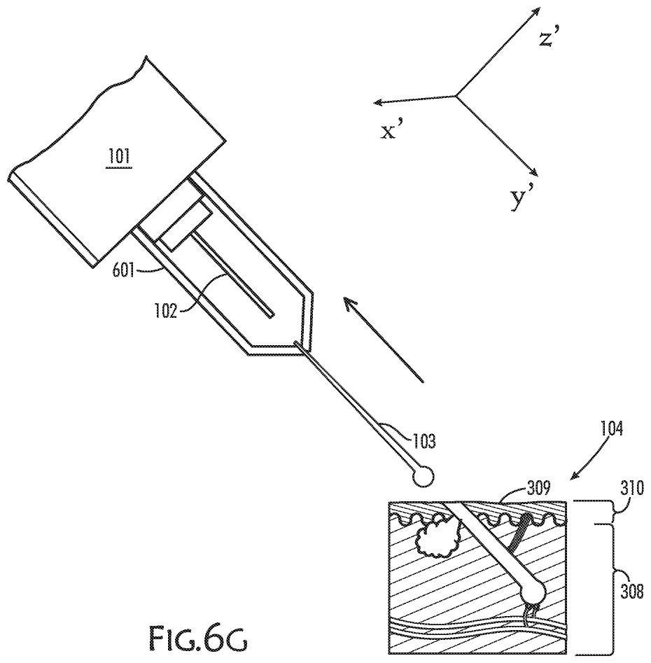

In another embodiment, a method of treating hair further comprises removing a hair. After the electrical, optical, and/or chemical treatment is provided at step 1203, in some cases, a hair is removed. For example, a hair 103 associated with a target, such as a hair follicle 302, a target hair component, and/or an additional target component, can be removed from the skin 104. In some cases, the hair 103 is removed using robotic tweezers 601, as shown for example in FIGS. 6A-6G. Tweezers 601, for example, can grasp the hair 103 and extract the hair from the dermal layer 308 by plucking. In other embodiments, the hair is removed by virtue of the treatment itself. For example, in some embodiments, removing a hair includes heating and/or galvanizing the hair 103, resulting in irreparable damage to the hair, such that the patient's body naturally removes the hair by natural reabsorption of the damaged hair.

In another embodiment, a method of treating hair further comprises imaging a target at 1201 of FIG. 12. For example, imaging a target comprises imaging a hair follicle, a target hair component, and/or an additional target component on the skin of a patient. In some embodiments, imaging a target comprises using one or more imaging devices 105, such as cameras, sensors, and/or systems thereof, to acquire data related to the target. In some cases, the one or more imaging devices further includes one or more pressure sensors.

Imaging devices 105, in some embodiments, provide detailed information about the target, including before providing treatment, while providing treatment, and/or after providing treatment. For example, imaging a target at 1201 comprises acquiring data related to the width, length, diameter, lean, directional growth, depth into the skin, and/or other characteristics or features of the target either before, during or after treatment. In some cases, data related to the location and/or spatial orientation of the target is collected in reference to a reference marker, such as the surface of the skin and/or other artificial reference marker placed around or near the target.

Imaging a target at step 1201, in some embodiments, occurs prior to positioning the one or more needles in a treatment position at step 1202 such that data acquired from the imaging devices informs the positioning of the one or more needles. For example, imaging a target at step 1201 can include determining the location and/or spatial orientation of the target to position the one or more needles at step 1201. In other embodiments, imaging a target at step 1201 occurs during or after providing electrical, optical, or chemical treatment at step 1203, such that data acquired from the imaging device informs the status of the target relative to the goals of the treatment method. Imaging a target at step 1201, in some cases, comprises acquiring real time images of the target, including real time images of the skin surface, hairs, or other skin components and information related to their 3-dimensional coordinates and/or orientation.

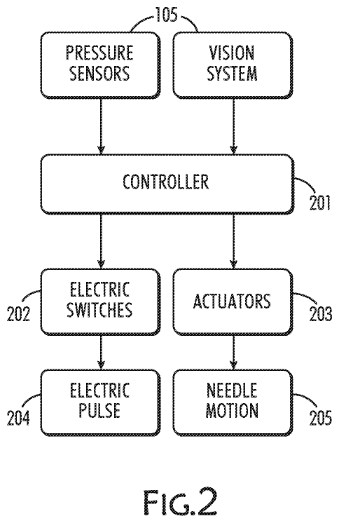

In other embodiments, a method of treating hair further comprises processing data received from the one or more imaging devices using a controller 201, as shown for example in FIG. 2. Processing, in some embodiments, comprises interpreting the data acquired from imaging devices into executable functions and/or instructions. For example, a controller 201 comprising hardware and/or software can process data related to the target, such as the location and/or spatial orientation, into instructions for one or more actuators 203 to move or position the one or more needles 205 in the treatment position. The one or more actuators 203, in some embodiments, comprise one or more mechanical actuators. In other embodiments, the one or more actuators comprise or define a 6-axis robot.

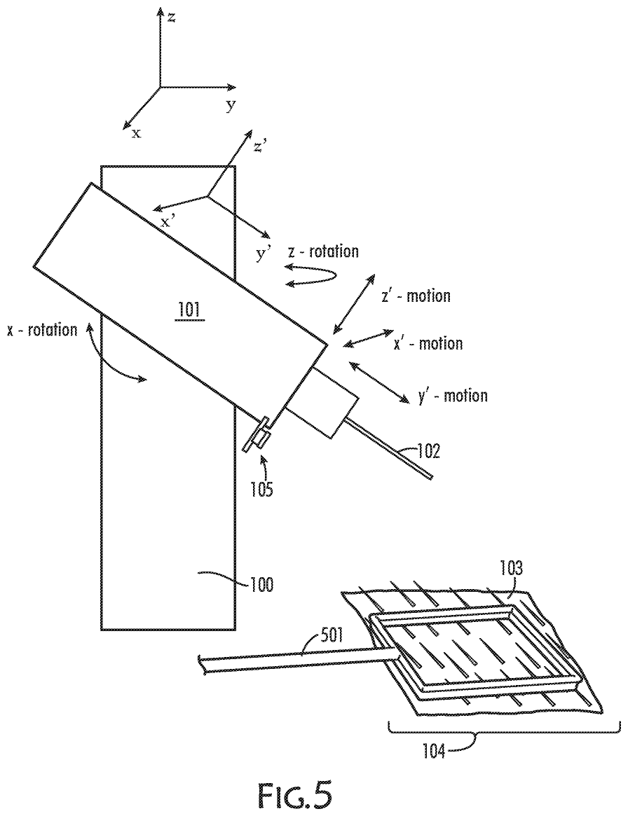

In some cases, a method further comprises attaching an applicator 501, as shown in FIG. 5, to the skin prior to imaging the target and/or prior to providing electrical, optical, or chemical treatment. The applicator 501, in some instances, is attached to the skin with an adhesive positioned on the bottom side of the applicator 501. In other instances, the applicator 501 is attached to the skin with a vacuum or other suction mechanism, such as suction cups present on the bottom side of the applicator. An applicator 501, in some cases, comprises a rectangular frame. A rectangular frame, in some embodiments, is relatively flat, having a thickness of less than 1 cm.

The applicator frame, in some instances, comprises additional markings and/or components, such as sensors on the top side of the frame that are identifiable by the imaging devices, including the cameras, sensors, and/or systems thereof. In some cases, the applicator frame itself, additional markings, and/or sensors of the applicator act as a reference point for the one or more imaging devices.

### II. Systems for Treating Hair

In another aspect, systems for treating hair are described herein, which are capable of performing the methods previously described in Section I. For example, systems described herein are can perform one or more methods of treating hair on human subjects or patients, as described in Section I. Additionally, systems for treating hair include systems for treating hair follicles, hair components, and/or sebaceous glands located on the skin of a patient. Thus, systems for treating hair can further include systems for treating hair on various locations of a patient's body. Systems for treating hair, as described herein, can include systems for treating any one or more macrostructures on or within the dermal layer wherein hair grows. For example, systems for treating hair includes systems for treating the hair itself, hair follicle, papilla, hair matrix, root sheath, hair bulb, hair bulge, infundibulum, arrector pili muscle, sebaceous gland, apocrine sweat gland, hair follicle receptor, skin surface, dermal layer of the skin, region of a hair follicle adjacent to a sebaceous gland, and/or other related macrostructures identifiable on or within a region of skin wherein hair resides. Furthermore, any one or more of these macrostructures can be a target of one or more of the systems described herein.

A system, in some embodiments, is a robotic electrolysis system for treating hair of a patient. A robotic electrolysis system, as described herein, can be utilized to perform any one or more of the methods described in Section I. Various components and/or elements of systems described herein are described in further detail in Section I.

A robotic electrolysis system comprising a robot and one or more controllers. In some cases, the robotic electrolysis system comprises a 6-axis robot, whereas in other cases, the robotic electrolysis system comprises a robot having multiple motors and multiple mechanical actuators. In some embodiments, a system further comprises a vision system and additional components, including an electrolysis needle base 101 and one or more needles 102 for delivering treatment, such as an electrolysis needle 700, optical fiber 901, and/or cannula 1101.

The controller of a robotic electrolysis system described herein comprises software and/or hardware for controlling components of the system. The controller receives data acquired by components of the robotic electrolysis system, processes the data, and interprets the data into executable instructions and/or functions. Importantly, the controller enables and regulates fluid rotation and movement on and around specific axes of robotic electrolysis system. The rotation and movement on and around specific axes allows the robotic electrolysis system to position itself and/or its components and perform one or more of the methods described in Section I.

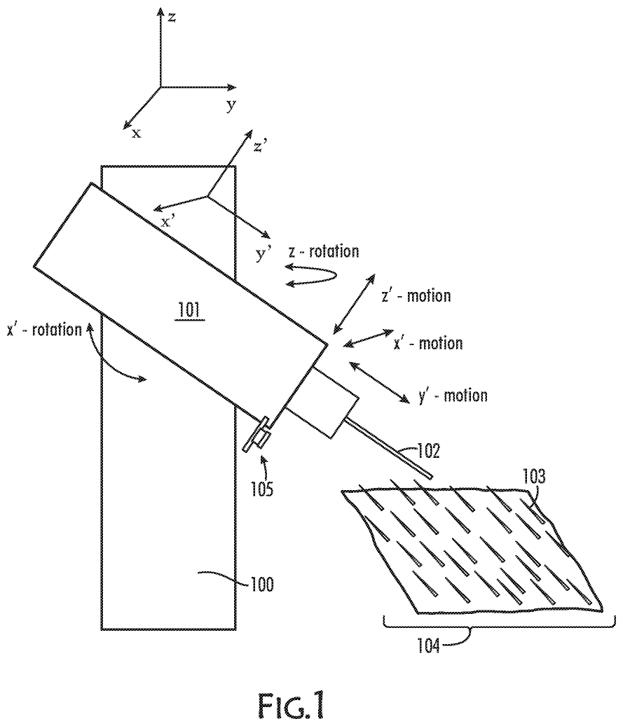

The robotic base 100, as shown in FIG. 1, in some embodiments, comprises one or more motors and one or more mechanical actuators. Each motor and/or actuator coordinates movement of the robot base around or along a specific axis identifiable in a representative Cartesian coordinate system. For example, as shown in FIG. 1, rotation around and movement along each of the xyz-axes of the robot base are performed by one or more motors and/or actuators.

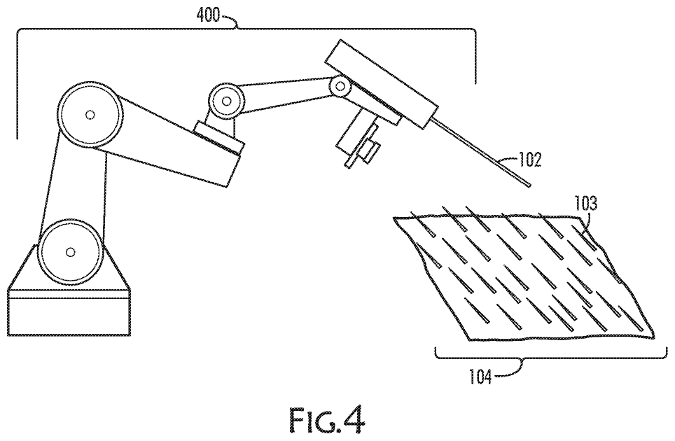

A robotic electrolysis system, in other embodiments and as shown in FIG. 4, can alternatively comprise a 6-axis robot 400 having six articulating joints to coordinate the movement of the robot in a 3-dimensional space. As shown in FIG. 4, the 6-axis robot 400 incorporates all six of the xyz- and x′y′z′-axes of the robotic base 100 and electrolysis needle base 101 of FIG. 1. Even still, the 6-axis robot 400 is configured to receive the one or more needles 102 to perform one or more methods of Section I.

In other embodiments, a robotic electrolysis system described herein comprises one or more additional components, including an electrolysis needle base 101 and one or more needles 102. In some embodiments, the electrolysis needle base 101 is connected to the robotic base 100 of the robotic electrolysis system. Wherein the robotic electrolysis system comprises a 6-axis robot 400, the electrolysis needle base 101 is connected to the terminal end of the 6-axis robot 400 opposite of its base, as shown in FIG. 4.

The electrolysis needle base 101, in some embodiments, comprises one or more motors and one or more actuators. Similar to the robotic base 100, each motor and/or actuator of an electrolysis needle base 101 coordinates movement of the electrolysis needle base 101 around or along specific axes of a representative Cartesian coordinate system. For example, as shown in FIG. 1, rotation around and movement along each of the x′y′z′-axes of the electrolysis needle base 101 are performed by one or more motors and/or actuators.

The electrolysis needle base 101, in other embodiments, is configured to receive at least one or more needles 102. For example, an electrolysis needle base 101, in some cases, comprises a mounting mechanism for positioning and securing the one or more needles 102 to the electrolysis needle base 101. The mounting mechanism can include a slot, hole, cavity, or other suitable receptacle for positioning and securing a needle to the electrolysis needle base 101.

In other embodiments, the electrolysis needle base 101 comprises one or more additional features or components. For example, an electrolysis needle base can, in some cases, further comprise tweezers 601 or tweezer-like arm structures, as shown in FIG. 6. The tweezer arms, in some cases, can extend from the body of the electrolysis needle base 101. For example, a proximal end of the tweezer arms flank a proximal end of the needle 102 with the arms of the tweezers 601 extending outward from the electrolysis needle base 101 and in parallel with the needle 102. In some embodiments, the tweezers 601, particularly when in use, can extend beyond the distal end of the needle 102. In other embodiments, the tweezer arms have a medial bend at their distal end so that when the tweezer arms are extended beyond the needle 102 and pinched together, the needle 102 remains undisturbed in its position as the distal ends of the tweezer arms meet, as shown in FIGS. 6E and 6F. Still in other embodiments, the tweezers 601 can retract into the electrolysis needle base 101, as shown in FIG. 6G. The retraction can occur when the tweezer arms are pinched or open. For example, the tweezers 601 can be deployed when necessary for use by the robotic electrolysis system. The tweezers 601 can be retracted upon securing a target, such as a hair 103, between the pinched tweezer arms. In another example, the tweezers 601 can retract, in some instances in their entirety, when the tweezer arms are open and not pinched.

In some embodiments, a robotic electrolysis system comprises a needle 102. The needle 102 of a robotic electrolysis system can vary depending on the type of treatment being delivered by the robotic electrolysis system. For example, the needle 102, in some embodiments, can be an electrolysis needle 700, an optical fiber 901, or a cannula 1101. In one example, a robotic electrolysis system providing an electrical treatment can use an electrolysis needle 700 as a needle 102. An electrolysis needle 700, in such instances, is connected to an electrical source, from which an electrical pulse or electrical current can travel through the electrolysis needle 700 providing energy to a target of the robotic electrolysis system. In another example, a robotic electrolysis system providing an optical treatment can use an optical fiber 901 as a needle 102. An optical fiber 901, in some instances, can be connected to laser light or a Broad Band intense pulse Light (BBL), from which a laser or pulse light can travel down the optical fiber 901 to a target of the robotic electrolysis system. In still another example, a robotic electrolysis system providing a chemical treatment can use a cannula 1101 as a needle 102. A cannula 1101, in some instances, is connected to a liquid source, such as sodium hydroxide, from which the liquid can travel down the cannula 1101 and out an opening 1102 on the distal end of the cannula 1101 to a target of the robotic electrolysis system. Other needles 102 can be used depending on the treatment type and the goals of the robotic electrolysis system, which are described in more detail of the methods of Section I.

The one or more needles 102, in some embodiments, is secured at a proximal end to the electrolysis needle base 101. The needle 102, for example, can extend beyond a distal end of the base away from the robotic electrolysis system and toward the target of the system, wherein a distal end of the needle 102 interacts with the target of the system. In some instances more than one needle 102, and or needle type, can be utilized by a robotic electrolysis system. For example, the one or more needles 102 can be interchangeable as needed to achieve the goals of the methods described in Section I.

In some embodiments, the needle 102 is an electrolysis needle 700. An electrolysis needle, in some embodiments, can have one or more conductive properties. For example, an electrolysis needle 700 can be formed from a conductive material. An electrolysis needle 700, in some embodiments, has a fully exposed conductive surface 701. Electrolysis needles having fully exposed conductive surfaces, as shown in FIG. 7A, can deliver energy to all exposed surfaces of the needle. In other embodiments, an electrolysis needle 700 has a partially exposed conductive surface 701. For example, a partially exposed electrolysis needle can have one or more coatings of a non-conductive material, such as silicone or other suitable polymer, to insulate the coated surfaces 702. Electrolysis needles having one or more coatings can have a conductive core and an insulating coating. In some embodiments, the coatings partially cover the electrolysis needle, leaving a portion of the conductive core exposed 701. The exposed portion 701 of the conductive core can be positioned along any cross sectional length of the electrolysis needle extending from its proximal end near the electrolysis needle base 101 to its distal end near the target. In some embodiments, the tip 703 of the conductive electrolysis needle 700 is exposed and the rest of the electrolysis needle is coated and/or insulated 702, as shown in FIG. 7B. In other embodiments, a midsection 704 of the conductive electrolysis needle 700 is exposed 701 and the rest of the electrolysis needle is coated 702, as shown in FIG. 7C. When a midsection 704 of an electrolysis needle 700 is exposed, the exposed surface 701 can include the entire circumferential surface area of the cross section of the electrolysis needle or only a portion of the circumferential surface area of the cross section of the electrolysis needle. Electrolysis needles having partially exposed conductive surfaces deliver energy to only the exposed surface 701 of the needle, rendering finer resolution of treatment delivery.

In some embodiments, a robotic electrolysis system described herein comprises one or more imaging devices 105. An imaging device can include, but is not limited to, any one or more of ultrasounds, x-rays, cameras, sensors, or systems thereof. Sensors, in some embodiments, are sensors of light. In other embodiments, an imaging device 105 includes devices capable of rendering 3-dimensional data and apply characteristics to the 3-dimensional data, such as hardness or toughness, temperature, or other characteristics.

In some embodiments, the one or more imaging devices 105 comprise a vision system. A vision system, as described herein, acquires 3-dimensional coordination data related to the shape, size, spatial orientation, and location of a target of the system relative to a reference marker, such as the surface of the skin. Thus, for example, a vision system can provide 3-dimensional spatial mapping of a target, such as a hair, a hair bulge, a hair bulb, a hair follicle, or a sebaceous gland, and its identifiable surroundings in one or more frames of the vision system. The vision system can use any electromagnetic wavelength necessary to acquire information or data related to the spatial coordinates of the target. For example, in some cases, optical coherence tomography or high-frequency ultrasound can be utilized within a vision system. A vision system, in some embodiments, can further comprise additional components, such as a light source. A light source, in some cases, can illuminate the target and/or its surroundings within one or more frames of the vision system, thereby enhancing the data acquisition processes of the vision system. The vision system can be operatively connected to the controller, and the controller can control the operation of the vision system.

A vision system, in some embodiments, is attached to the robotic electrolysis base. Alternatively, a vision system, in other embodiments is attached to the electrolysis needle base. In still other embodiments, a vision system, can be attached to a component or member of a 6-axis robot. Additionally, a vision system, is some instances, is automated such that data acquired from the vision system is automatically processed by the controller into executable functions.

Additionally, imaging devices 105 of a robotic electrolysis system can include other sensors operably connected to the controller, such as pressure, temperature, infrared, gyroscopic, or accelerometer sensors. For example, a system, in some instances, can have one or more pressure sensors. Pressure sensors, in some cases, provide data related to the environment of the system's target, such as the toughness, hardness, stiffness, or pliability of the skin. For example, pressure sensors, in some embodiments, provide data to the robotic electrolysis system as the needle enters the skin, so the controller can control and adjust the amount of force provided by the robotic electrolysis system to the needle as the needle enters the skin.

Other sensors can also be used to provide data and/or feedback to the robotic electrolysis system, which in some cases, occurs in real time. The sensors can likewise also be operatively connected to the controller 201. Data and/or feedback from these sensors can be processed by the controller 201 into executable functions and/or functional corrections. Furthermore, data from the sensors, including pressure sensors, can be combined with data with other light and/or ultrasonic sensors by the controller, such as the vision system, to provide a comprehensive data set to the robotic electrolysis system for processing executable functions, such as positioning, advancing, and/or rotating one or more of the robot base 100, electrolysis needle base 101, or needles 102.

In other embodiments, a system further comprises an applicator 501 configured to be attached to or disposed on the skin, as seen for example in FIG. 5. An applicator 501, for example, can comprise a rectangular, square, circular, oval, polygonal, or other shaped frame, wherein the frame is attached to the skin. The applicator 105, in some embodiments, is relatively flat and has a thickness less than 1 cm, less than 0.75 cm, less than 0.50 cm, or less than 0.25 cm. In some instances, the frame is attached to the skin with an adhesive. An adhesive, in some cases, is positioned on the bottom side of the frame. In other instances, the frame is attached to the skin with a vacuum or suction mechanism, such as suction cups. An applicator frame, in other embodiments, can have markings on the top side of the frame that are identifiable by the vision system and/or other sensors of the system. In some cases, the applicator frame and/or the markings on the top side of the applicator frame act as a reference point for the vision system and/or other sensors of the system.

The skilled artisan would understand that various modifications can be made to the above described systems and methods without departing from the scope and objectives of the invention. For example, although specific configurations of systems are described above and depicted in the figures, other robotic electrolysis systems configured treat hair can benefit from embodiments of the systems and methods described herein. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

Various implementations of systems and methods have been described, and exemplary embodiments are described below in fulfillment of various objectives of the present disclosure. It should be recognized that these implementations are merely illustrative of the principles of the present disclosure. Numerous modifications and adaptations thereof will be readily apparent to those skilled in the art without departing from the spirit and scope of the present disclosure. For example, individual steps of methods described herein can be carried out in any manner not inconsistent with the objectives of the present disclosure, and various configurations or adaptations of methods and systems described herein can be used.

### Example 1

### Robotic Electrolysis System

FIG. 1 shows a robotic electrolysis system according to one embodiment described herein. The system illustrated in FIG. 1 includes multiple components, as listed below.

The robotic electrolysis system includes a robotic base 100, a needle base 101, and an electrolysis needle 102. To characterize the motion of the robotic electrolysis system, the Cartesian coordinate system xyz associated with and above the robotic base 100 is shown in the FIG. 1. In addition, a second Cartesian coordinate system x′y′z′ associated with the needle base 101 is also shown to separate the movements of the two components with respect to one another.

The robotic electrolysis system further includes motors and actuators (not shown) to move specific parts or components of the system relative to other parts or components of the system:

1. Actuator #1 rotates the needle base 101 relative to the robot base 100 around the z-axis.

2. Actuator #2 rotates the needle base 101 relative to the robot base 100 around the x′-axis. These two rotations are used together to orient the needle base and the needle and to align the needle with respect to the orientation of the hair.

3. Actuator #3 moves the needle base 101 along the z′-axis

4. Actuator #4 moves the needle base 101 along the x′-axis.

These two motions are used to position the needle base 101 and needle 102 towards the target hair 103.

5. Actuator #5 is used to move the needle relative to the needle base along the y′-axis. This motion is used to insert the needle 102 into the skin 104 along the hair follicle and to remove the needle 102 from the skin 104 after the treatment.

The robotic electrolysis system can also include a vision system 105.

For example, a vision system 105 can include one or more cameras (or other imaging devices) or a system of cameras (or other imaging devices) (not shown) and an illumination source(s) (not shown) to image the skin surface and the hairs 103 to determine their positions. Optical Coherence Tomography (OCT) or high-frequency ultrasound (HFUS) can also be used to image the skin surface and the hairs and to determine the orientations and positions of the hairs. Such components can form or define a “vision system” or subsystem or component of the overall system. It is to be understood that such a “vision system” can include one or more cameras and optionally one or more illumination sources (e.g., light sources). The vision system 105 can be attached to the needle base 100 or the robot base 100. Moreover, the vision system 105 is generally capable of capturing/mapping the 3-dimensional coordinates of the skin 104 and the hairs 103. Further, these cameras or system of cameras or OCT or HFUS components described above can form part of the vision system 105 of the robotic device.

The robotic system can also include pressure sensors to measure the pressure associated with the insertion of a needle 102 into the skin 104. Feedback from these sensors can then be used to better estimate spatial orientation of the hair follicle, once the needle 102 is inside the skin 104.

The robotic system can also include/incorporate electronic components that are currently found in existing electrolysis hair removal devices, such as devices using direct current supply and alternating current supply of radio frequency (RF). Such current supplies can provide electric current to the electrolysis needle of a system described herein.

FIG. 2 is a block diagram schematically describing the operation of a robotic electrolysis system according to one embodiment described herein. In this embodiment, the robotic procedure begins by acquiring the data from the imaging devices 105, such as a vision system and pressure sensors to perform the necessary calculations to identify the location and orientation of the hair, as in step 1201 of FIG. 12. The controller 201 uses the data to perform needle motion 205, via actuators 203, to move the needle base 101 and needle 102 to the proper location and to orient the needle appropriately, as in step 1202 of FIG. 12. The controller 201 also uses the data to activate electric switches 202 to deliver energy, in the form of an electric pulse 204, to the robotic needle 102, as in step 1203 of FIG. 12.

### Example 2

### Methods of Treating Hair Using a Robotic Electrolysis System

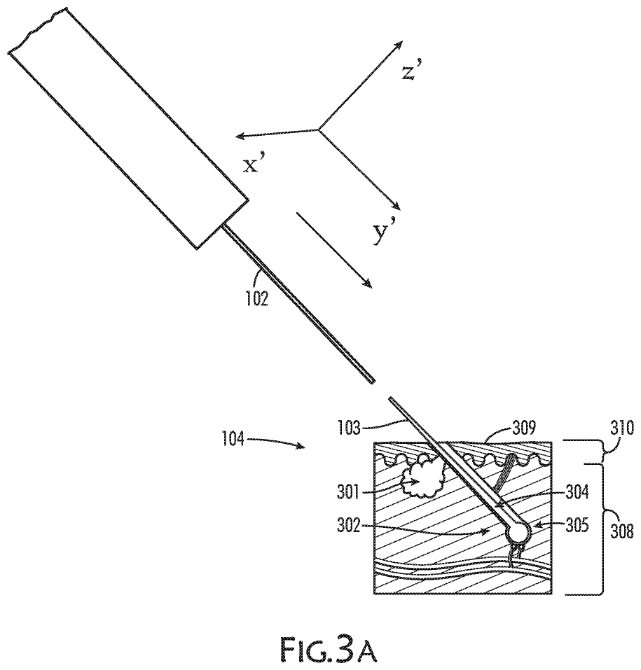

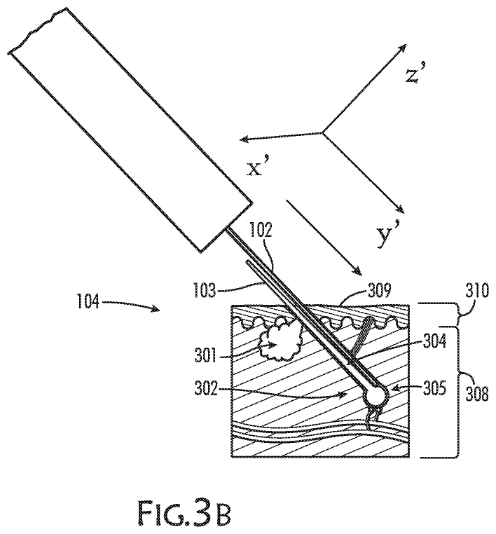

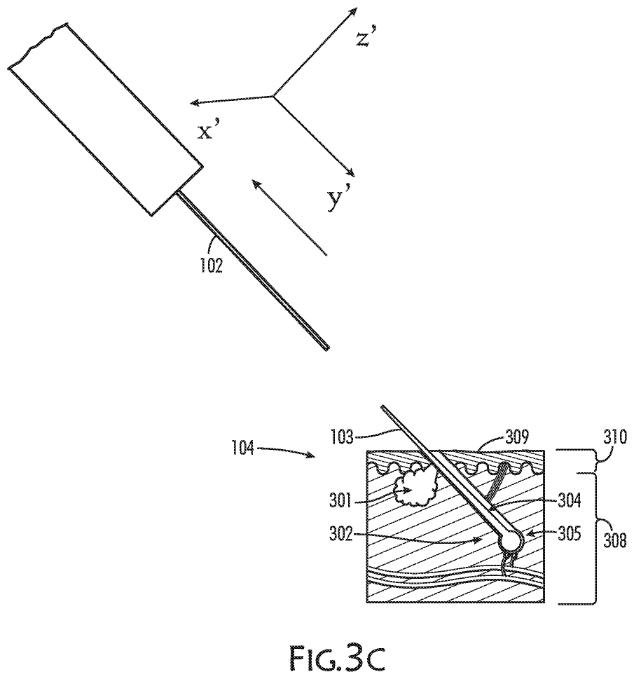

A robotic electrolysis hair removal procedure or method, in some cases, comprises or consists of the following steps:
1. Prior to the procedure, the treated hair can optionally be trimmed to 0.5 mm, 1 mm, 1.5 mm, or 2 mm in length. This will allow a clear camera view and will make the hair recognition procedure easier.
2. The robotic device is then positioned at a working distance next to the skin surface 309 with respect to the hairs targeted for removal (i.e., target hairs).
3. A vision system takes real time images of the skin surface 309 and hairs 103 and determines the hairs 3-dimensional coordinates and orientation within a target area on the skin surface 309.
4. Using these coordinates, the system rotates the needle base to align the needle base parallel to the hair growth direction, as shown in FIG. 3A.
5. The controller then calculates the hair coordinates in a new x′y′z′ coordinate system based on the real time images.
6. The system aligns the needle base to the target hairs by moving the needle base along the x′- and z′-axes.
7. The system then moves the needle along the y′-axis to insert the needle into the skin, parallel to the hair follicle 302.
8. After the needle penetrates the skin along the hair shaft by the appropriate distance, the system applies energy or other treatment (e.g., electric current (DC and/or RF), laser light, or even chemical treatment. However, in some embodiments are described herein with reference to “electrolysis” in particular, it is to be understood that the electrolysis steps described herein can generally be replaced with other treatment steps (such as laser light, BBL, or chemical treatment steps) to treat the hair follicle, as shown in FIG. 3B.
9. The system then removes the needle 102 along the y′-axis from the hair follicle 302.
10. The system then repeats the steps onto the next target hair follicle 302.

It is to be understood that the same system procedures would be applicable to other hair structures or to the sebaceous glands 301.

In certain cases, the procedure can be simplified since the majority of hairs in a given area of skin often grow in a similar direction. In those cases, the needle base angular orientation does not have to change after each hair treatment procedure. Only Cartesian coordinates (x′ and z′) of the needle base will need to change in order to move the needle base from one target hair to the next.

In other cases, feedback from the pressure sensors can be necessary to correct the angular orientation of the needle base, to perform the needle insertion to reduce collateral damage to the skin.

As shown in FIG. 4, a 6-axis robot 400 or robotic system can be used instead of the system of 5 actuators described above, although again, systems having 2, 3, or 4 actuators are also contemplated.

Due to the operation of the system described above, an applicator 501 can be applied to the skin, before the robotic movement starts, to help the system better locate and orient the needle. One exemplary applicator 501 is illustrated in FIG. 5. The applicator 501 shown in FIG. 5 is a rectangular frame attached to the skin surface (with vacuum suction or some other method) and serves dual functions: to provide a better visual reference for the vision system; and to ensure that the target skin area is not moving relative to the robot base 100.

### Example 3

### Method of Treating and Removing Hair Using a Robotic Electrolysis System

An aspect of permanent hair removal/reduction is the destruction of the hair structures. Such destruction prevents future growth. The immediate removal of the hair does not affect regrowth. However, after treatment is performed as described herein, the treated hairs can be manually removed. A robotic system described herein can include additional components for removing the hair from the skin after treatment, such as shown in FIGS. 6A-6G, for instance. These components can include tweezers 601, located at the needle base 101, which can also be moved by additional actuators. In the case of hair removal, the system will perform the removal as follows:

System is configured to determine the angular and Cartesian coordinates of the hairs and adjust the needle base 101 position accordingly. The needle 102 moves into the hair follicle 302—FIG. 6A; energy is applied to the needle—FIG. 6B; the needle moves out of the hair follicle 302—FIG. 6C; tweezers 601 are moved towards the hair 103—FIG. 6D; tweezers 601 are closed, grabbing the hair—FIG. 6E; and tweezers 601 are moved back, removing (e.g., plucking or pulling) the treated hair from the skin 104—FIG. 6F

### Example 4

### Types of Electrolysis Needles

Different types of needles 102 can be used in a robotic hair removal procedure described herein. The different types of electrolysis needles 700 include full surface exposure to partially exposed surfaces. FIG. 7A shows a standard electrolysis needle 700 that has its electrically conductive surface 701 fully exposed. At the insertion of this needle into the hair follicle and at the application of the electric current the full surface of this needle will deliver electric current.

FIG. 7B shows a conductive needle covered with a non-conductive coating 702 along its entire surface except for at the needle tip 703. FIG. 7C shows a conductive needle covered with a non-conductive coating 702 along its entire surface except for a small midsection 704 region of the needle surface located at a certain distance from the needle tip 703, wherein the electrically conductive surface 701 is exposed. At the insertion of the needles from FIG. 7B or FIG. 7C into the hair follicle and at the application of the energy, only the exposed surfaces 701 of the needle will deliver energy. Thus, the area of the hair follicle next to the exposed part of the needle will be exposed to the majority of the energy resulting in targeted treatment.

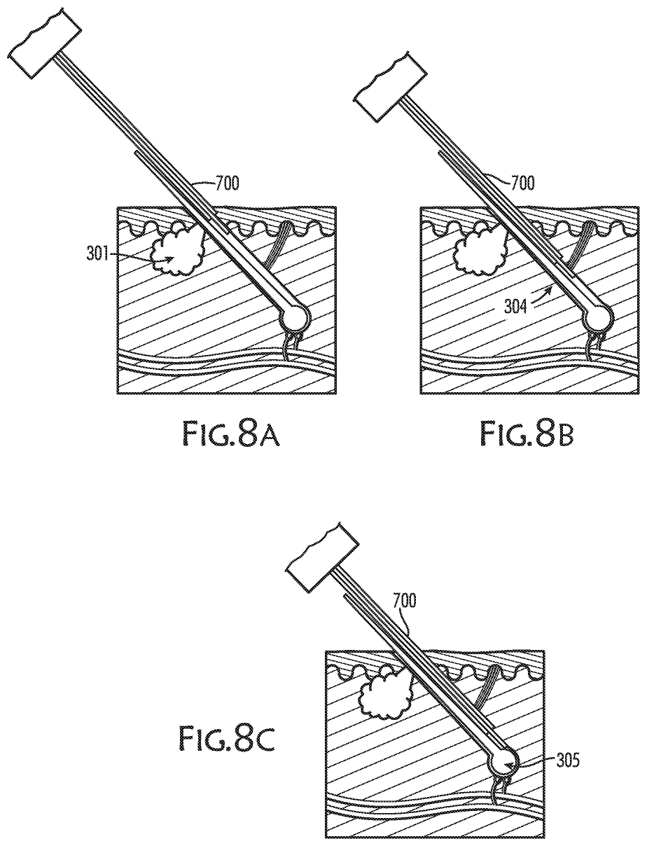

FIG. 8 shows three possible destruction targets in the hair follicle treated by the partially-exposed needle: A—sebaceous gland 301, B—hair bulge 304, C—hair bulb 305. While the sebaceous gland 301 can be the destruction target for the treatment of acne or other skin conditions related to the sebaceous gland activity, the hair bulge 304 and hair bulb 305 can be the primary targets for hair reduction/removal.

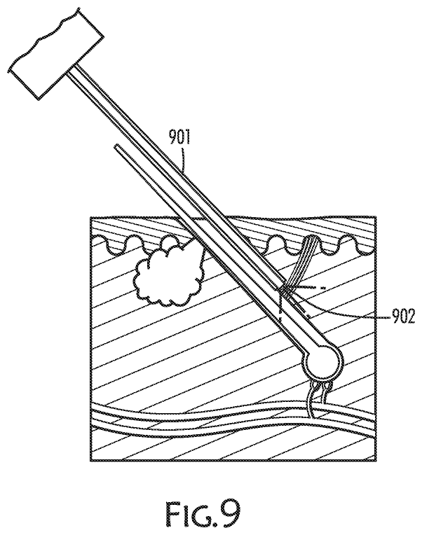

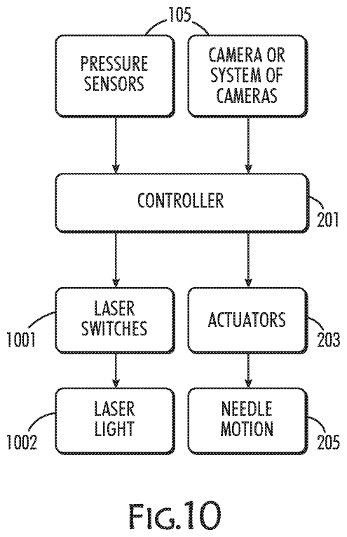

FIG. 9 shows a robotic optical fiber 901, which can be used instead of the electrolysis needle 700 in a robotic needle system described herein. The robotic optical fiber system is used to treat the target in the hair follicle 302. In this system, the optical fiber 901 is inserted into the hair follicle 302, instead of the electrolysis needle 700 in the case of the robotic electrolysis system. When the optical fiber 901 is inserted into the hair follicle 302 at the appropriate depth the light is turned on by the robotic optical fiber system, which exits the optical fiber tip 902 and treats the hair follicle 302 and the skin 104 around it.

### Example 5

### Method of Treating Hair Using a Robotic Electrolysis System Having an Optical Fiber

FIG. 10 is a block diagram of the robotic optical fiber system. A robotic system controller 201 reads the data from the imaging devices 105, such as a camera or system of cameras and pressure sensors, performs calculations and sends the signals to actuators 205 to perform needle motion 205, such as moving the needle base 102 and the optical fiber 901. The controller 201 further performs calculations and sends signals to the laser switches 1001 that send the laser light 1002 into the optical fiber 901.

The hair follicle treatment procedure with an optical fiber 901 is similar to the treatment procedure with the electrolysis needle 700. As described in FIGS. 1, 3-6, 8 and the accompanying text, an optical fiber 901, instead of an electrolysis needle 700, can be used to damage the hair structures thereby resulting in hair reduction/removal.

### Example 6

### Method of Treating Hair Using a Robotic Electrolysis System Having a Cannula

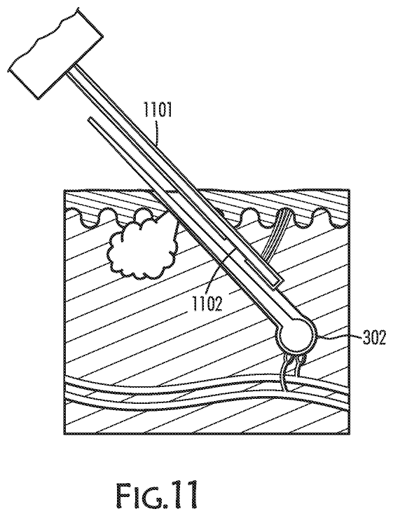

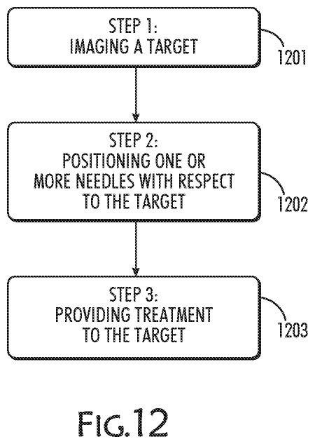

FIG. 11 demonstrates how a robotic cannula 1101 can also be used for hair reduction/removal. A robotic cannula system is used to treat the target(s) in the hair follicle 302 by releasing a treatment liquid in the cannula 1101 at the appropriate depth to the appropriate target in the hair follicle 302. One example of a treatment liquid includes sodium hydroxide (NaOH, also known as lye and caustic soda), which can destroy the hair follicle 302. In this system, the cannula 1101 is inserted into the hair follicle 302, instead of the electrolysis needle 700. When the cannula 1101 is inserted into the hair follicle 302 at the appropriate depth, the liquid is dispensed into the hair follicle 302 and it treats the hair follicle 302, and surrounding tissues and structures.

1. A method of treating hair on a patient's skin, the method comprising:
imaging a target using one or more imaging devices;
positioning one or more needles in a treatment position with respect to the target; and
providing electrical, optical, or chemical treatment to the target using the one or more needles,
wherein the target is a hair follicle, a hair component, or a skin component.

2. The method of claim 1, wherein the method further comprises removing a hair associated with the target.

3. The method of claim 1, wherein the one or more imaging devices comprise one or more cameras or systems of cameras.

4. The method of claim 1, wherein the one or more imaging devices comprise one or more pressure sensors.

5. The method of claim 1, wherein the method further comprises processing data received from the one or more imaging devices using a controller comprising hardware and software.

6. The method of claim 5, wherein the processing data further comprises directing one or more actuating components to position the one or more needles in the treatment position.

7. The method of claim 6, wherein the one or more actuating components comprise one or more mechanical actuators.

8. The method of claim 6, wherein the one or more actuating components comprise a 6-axis robot.

9. The method of claim 1, wherein the one or more needles comprise one or more electrolysis needles.

10. The method of claim 9, wherein electrical current is applied to the target using the one or more electrolysis needles.

11. The method of claim 1, wherein the one or more needles comprise one or more optical fibers.

12. The method of claim 11, wherein a laser light or Broad Band intense pulse Light (BBL) is applied to the target using the one or more optical fibers.

13. The method of claim 1, wherein the one or more needles comprise one or more cannulas.

14. The method of claim 13, wherein a chemical treatment species is applied to the target using the one or more cannulas.

15. The method of claim 1, wherein the method further comprises attaching an applicator to the skin prior to providing the electrical, optical, or chemical treatment.

16. The method of claim 15, wherein the applicator is attached to the skin with an adhesive.

17. The method of claim 17, wherein the applicator is attached to the skin via vacuum or suction.

18. The method of claim 2, wherein the hair is removed using robotic tweezers.

19. The method of claim 1, wherein the skin component is a sebaceous gland or a region of the target hair follicle adjacent to the sebaceous gland.

20. The method of claim 1, wherein the hair component is a hair bulb or a hair bulge.
